(19) United States
(12) Patent Application Publication
(10) Pub. No.: US 2006/0155306 A1
(43) Pub. Date: Jul. 13, 2006

(54) Pimple removal utensil

(76) Inventor: Hyung-Chul Kang, Seoul (KR)

Correspondence Address: Frank Chau;Frank Chau & Asociates, 130 Woodbury Road, Woodbury, NY, 11797, US

(21) Appl. No.: 10/516,684

(22) PCT Filed: Jun. 4, 2003
(86) PCT No.: PCT/KR03/01101

§ 371 Date: Dec. 3, 2004

(30) Foreign Application Priority Data
Jun. 5, 2002 (KR) 20-2002-0017309
May 2, 2003 (KR) 10-2003-0028202

(51) Int. Cl. A61B 17/50

(52) U.S. Cl. 606/131

(57) ABSTRACT

A pimple removal utensil is provided in which pimples are easily squeezed open sanitarily with the pimples left untouched irrespective of size of the pimples. The pimple removal utensil includes a holder having an uneven surface which is formed to be easily held by the hand, and an auxiliary grip protruded perpendicularly with the uneven surface, and a pressing portion in which a first extension and a second extension are formed so that both extensions correspond to each other by opening one portion on the circumference of an insertion hole, and gradually narrowing the opened edges, and the bottom surface thereof is formed of a convex curved surface. A massage ball is further provided on the upper portion of the holder. The first and second extensions are made of a number of indents, which are gradually narrowed in correspondence to each other.

### TECHNICAL FIELD

The present invention relates to a pimple removal utensil, and more particularly, to a pimple removal utensil with which a pimple can be easily squeezed open through two extensions which are separated in each one end from each other and face each other.

### BACKGROUND ART

Pimples are pimpled in an adolescent age, and called symbols of youth. Pimples are pimpled because sebaceous glands are stimulated by the hormone starting to be secreted at the adolescent age, to promote sebums to be secreted, and then the sebaceous glands become corpulent to thus vividly secrete sebums. However, a disease of pimples is the most common skin disease, which can occur even in any one such as women in the thirties to forties, or men who suffer from much stress. Pimples occur in the face, neck, chest, etc, in which sebaceous glands are grouped together. Inflammation arising in the sebaceous glands attached to a hair pocket producing a hair generates pimples.

Pimples at the adolescent age are considered as temporary physiological phenomena. However, if pimples are not well treated, perpetual scars may be left.

Pimples are squeezed open by two nails or a tool such as a ball-point pen, in order to remove pimples. This is not hygienic, and may induce a secondary bacillus infection due to bacteria in the non-hygienic nails or tool. As a result, portions from which pimples have been squeezed fester to then leave scars.

Also, as shown in FIGS. 1, 2A and 2B, pimples can be removed by using a conventional pimple removal utensil formed of a holder 10, an intermediate portion 20, and a pressing portion 30. Here, an opening 32 formed in an insertion hole 34 of the pressing portion 30 is formed in a line. As a result, since pimples cannot be effectively pressed at their sides, they are not easily squeezed open, to thus cause a strong pressure on the pimples.

Meanwhile, a set of pimple removal utensils should be used according to the size of the insertion hole 34 relying upon the size of pimples. Also, since the bottom surface of the pressing portion 30 is formed flatly, pimples cannot be effectively squeezed open. Further, if a post-treatment is not performed after squeezing open pimples, hair holes are open and left alone.

### DISCLOSURE OF THE INVENTION

To solve the above problems, it is an object of the present invention to provide a pimple removal utensil for easily squeezing open pimples in which a pimple is positioned between two extensions which are opened in each one end thereof and is squeezed open by pushing the two extensions.

It is another object of the present invention to provide a pimple removal utensil for much more effectively squeezing pimples in which at least one indent formed on one side of each of tow extensions presses the side of a pimple alternately.

It is still another object of the present invention to provide a pimple removal utensil for easily squeezing pimples in which the bottom of the pressing portion is formed of a curved surface to thereby concentratively press a pimple.

It is yet another object of the present invention to provide a pimple removal utensil for further more effectively squeezing pimples in which two extensions get narrow gradually as it reaches their ends, and the side of a pimple can be more pressed as the two extensions are pushed.

It is yet still another object of the present invention to provide a pimple removal utensil in which an auxiliary grip is formed at the side of a holder to thereby prevent the pimple removal utensil from idly turning in a hand during removal of a pimple.

It is a further object of the present invention to provide a pimple removal utensil in which a massage ball is formed on the upper portion of a holder to thereby massage and close a hair hole, which is open after removal of a pimple.

To accomplish the above object of the present invention, there is provided a pimple removal utensil comprising: a holder; and a pressing portion having a first extension and a second extension which correspond to each other and are branched off and extended from the holder, and a pimple insertion hole formed inward the first and second extensions.

It is preferable that a number of indents are formed on the inner contacting surfaces of the first and second extensions, in a corresponding unevenness shape.

It is preferable that the bottom of the pressing portion is formed of a convex curved surface.

It is preferable that the interval between the first and second extensions becomes narrow gradually as it reaches from the insertion hole to the end.

It is preferable that the pimple removal utensil according to the present invention further comprises an auxiliary grip formed in the perpendicular direction from the holder.

It is preferable that the pimple removal utensil according to the present invention further comprises a ball-shaped massage ball formed on the upper portion of the holder.

The pimple removal utensil according to the present invention having the above-described configuration, can easily remove a pimple by gradually pushing the side of a pimple through the branched-off extensions.

### BRIEF DESCRIPTION OF THE DRAWINGS

The above and other objects and advantages of the present invention will become more apparent by describing the preferred embodiments thereof in more detail with reference to the accompanying drawings in which:

FIG. 1 is a perspective view showing a conventional pimple removal utensil;

FIG. 2A is a plan view showing a pressing portion in the conventional pimple removal utensil of FIG. 1;

FIG. 2B is a side view showing the pressing portion in the conventional pimple removal utensil of FIG. 1;

FIG. 3 is a perspective view showing a pimple removal utensil according to a first embodiment of the present invention;

FIG. 4 is a partially enlarged perspective view showing a pressing portion in the pimple removal utensil of FIG. 3;

FIG. 5A is a plan view showing a pressing portion in the pimple removal utensil of FIG. 3;

FIG. 5B is a side view showing the pressing portion in the conventional pimple removal utensil of FIG. 3;

FIG. 6 is a plan view showing a pressing portion in a pimple removal utensil according to a second embodiment of the present invention;

FIG. 7 is a perspective view showing a pimple removal utensil according to a third embodiment of the present invention; and

FIGS. 8A through 8D are sectional views showing states where pimples are squeezed open by using the pimple removal utensil according to the present invention.

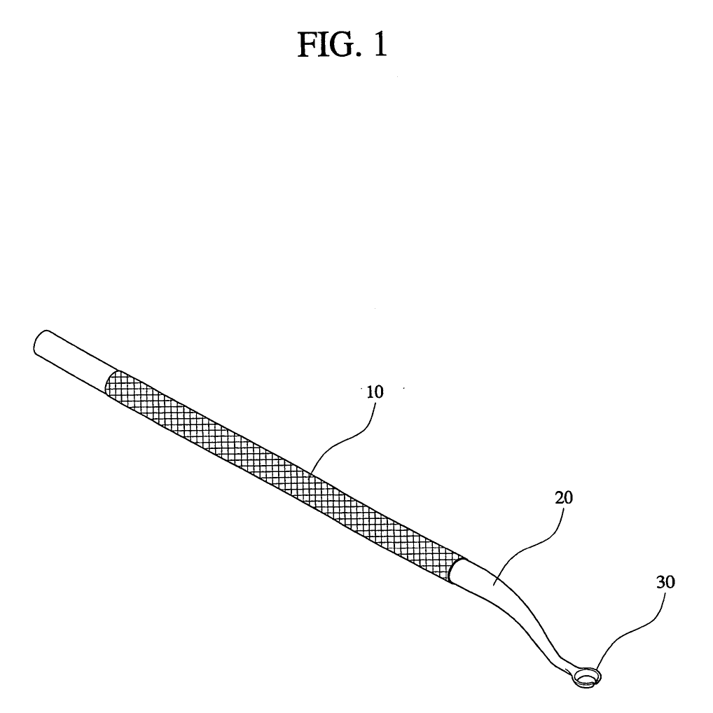

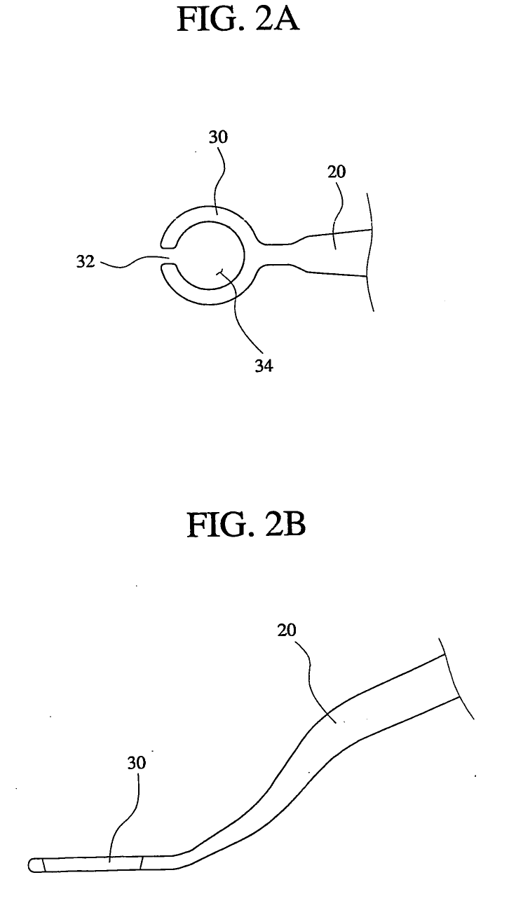

### BEST MODE FOR CARRYING OUT THE INVENTION

A preferred embodiment of the present invention will be described in detail with reference to the accompanying drawings.

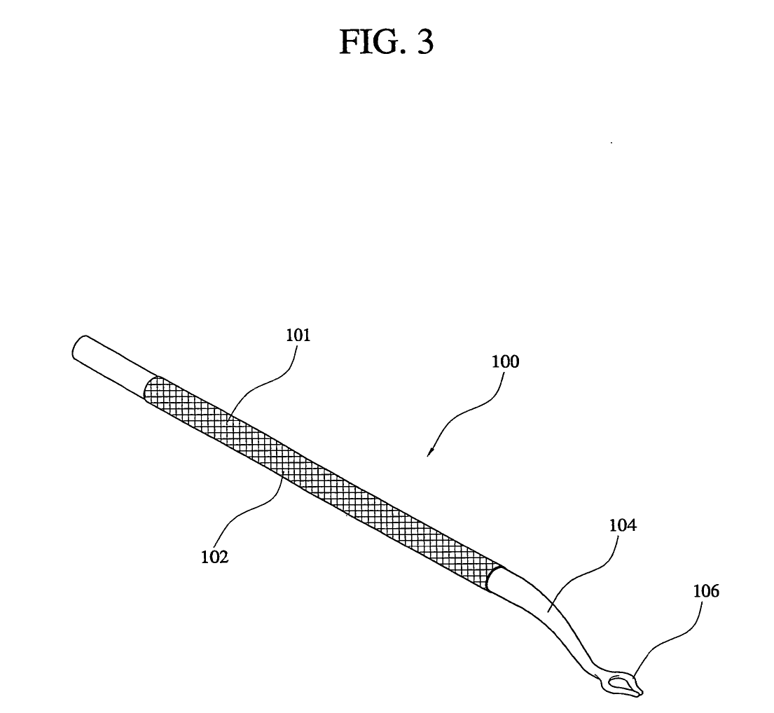

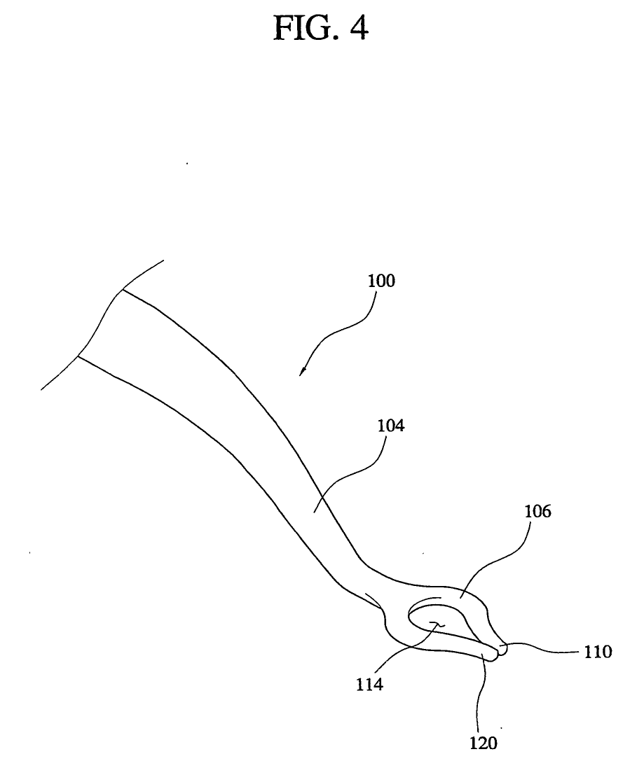

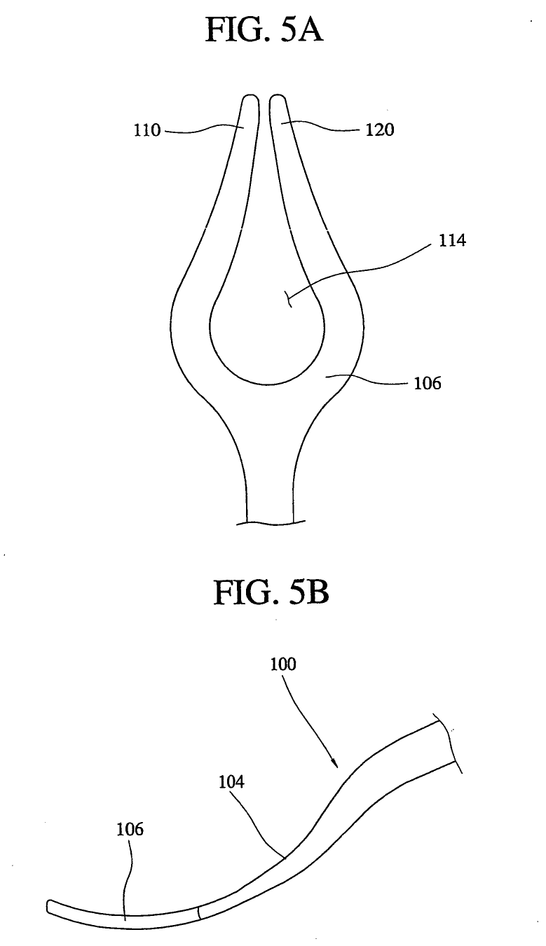

FIG. 3 is a perspective view showing a pimple removal utensil according to a first embodiment of the present invention. FIG. 4 is a partially enlarged perspective view showing a pressing portion of FIG. 3. FIG. 5A is a plan view of the pressing portion of FIG. 3, and FIG. 5B is a side view of the pressing portion thereof.

As shown in FIGS. 3 to 5B, a pimple removal utensil 100 includes a holder 101 and a pressing portion 106 largely.

The holder 101 is formed of an uneven surface 102 so that the holder 101 can be grasped well in a hand.

The pressing portion 106 is connected to an intermediate portion 104 to then be integrated with the holer 101, in which a first extension 110 and a second extension 120 are formed so that both extensions correspond to each other by opening one portion on the circumference of an insertion hole 114, and gradually narrowing the opened edges, and the bottom surface of the pressing portion 106 is formed of a convex curved surface.

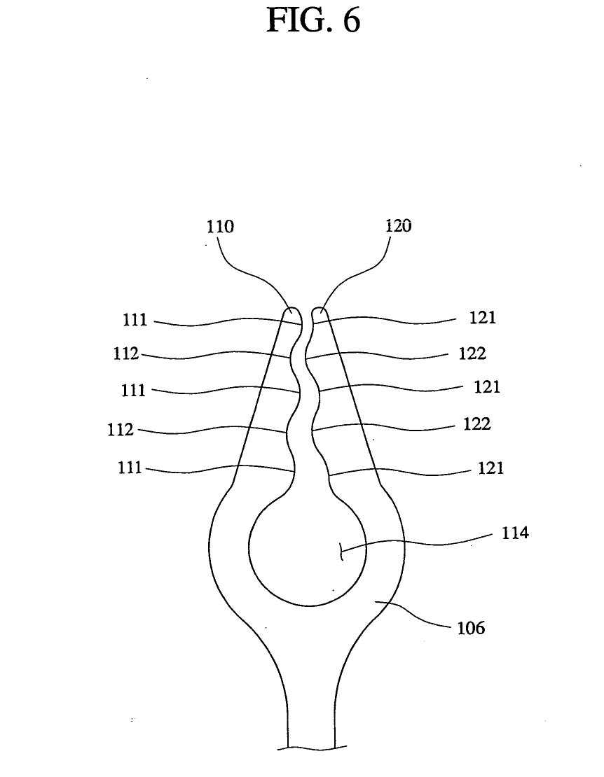

FIG. 6 is a plan view showing a pressing portion in a pimple removal utensil according to a second embodiment of the present invention.

In a pimple removal utensil 100 shown in FIG. 6, the inner contacting surfaces of a first extension 110 and a second extension 120 become narrow gradually, and thus a number of indents including a number of grooves 112 and 121 and a number of protrusions 111 and 122 are formed in a corresponding uneven shape. The bottom of the pressing portion 106 is formed of a convex curved surface. The number of grooves 112 and 121 and the number of protrusions 111 and 122 play a role of increasing a force pressing a pimple by means of the first and second extensions 110 and 120.

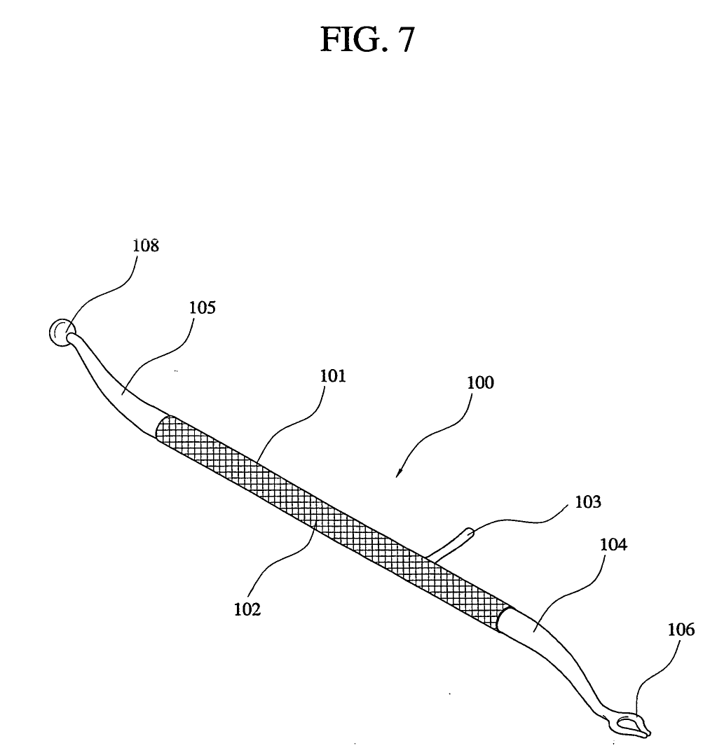

FIG. 7 is a perspective view showing a pimple removal utensil according to a third embodiment of the present invention.

A pimple removal utensil 100 shown in FIG. 7 according to the present invention includes a holder 101, a pressing portion 106, and a massage ball 108.

The holder 101 includes an auxiliary grip 103 formed perpendicularly with an uneven surface 102 in the lengthy direction of the holder 101 in order to prevent the holder 101 from idly turning in a hand.

The pressing portion 106 has the same structure as that of the first embodiment, but may be formed of the same structure as that of the second embodiment.

The massage ball 108 is formed of a ball-shaped structure on the upper portion of the holder 101, and is connected to a connection rod 105 to be integrated with the holder 101.

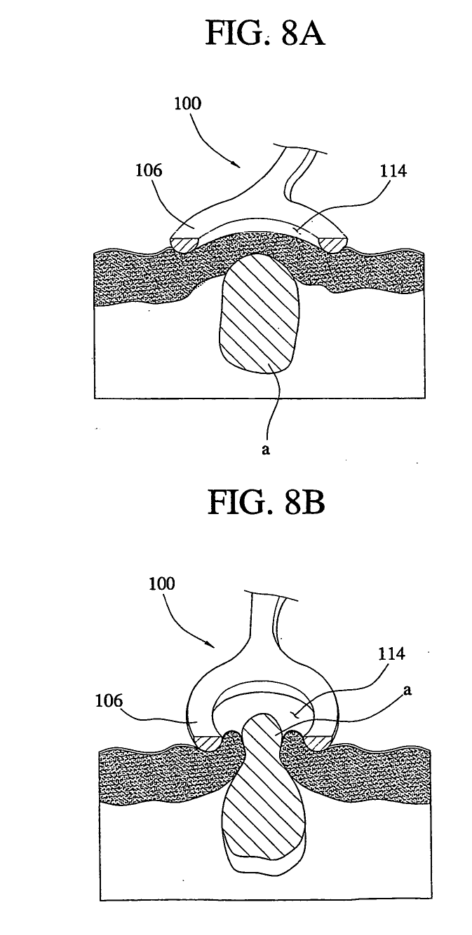

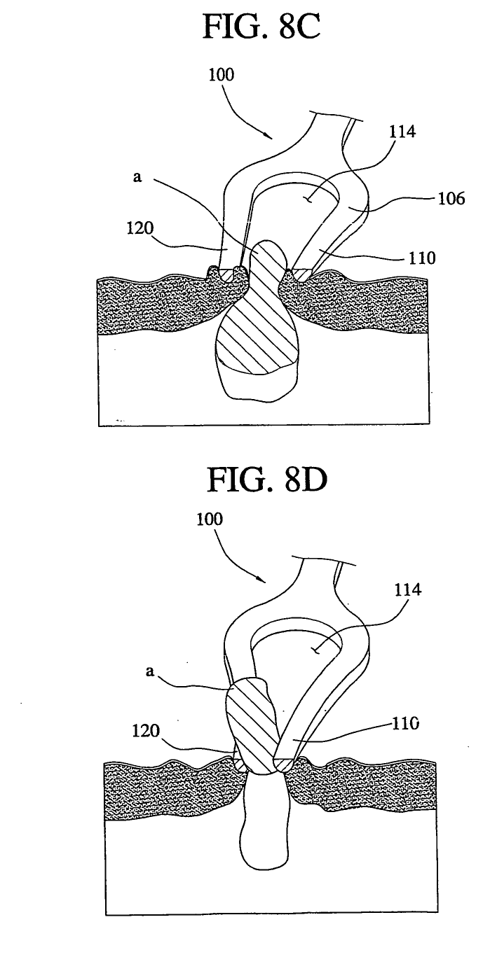

FIGS. 8A through 8D are sectional views showing states where pimples are squeezed open by using the pimple removal utensil according to the present invention.

In the pimple removal utensil 100 according to the present invention as shown in FIGS. 8A through 8D, the holder 101 is grasped with two fingers in which the uneven surface 102 of the holder 101 is grasped between the two fingers. As shown in FIG. 8A, the insertion hole 114 formed at the central portion of the pressing portion 106 is positioned on a pimple “a.” Then, as shown in FIG. 8B, the pimple removal utensil 100 is smoothly moved so that the opening portion formed between the ends of the first and second extensions 110 and 120 passes through along the convex curved surface of the bottom of the pressing portion 106 at the state where the pressing portion 106 has been pressed. Thus, when the bottom of the pressing portion 106 is moved at the state where the peripherals of the pimple “a” have been pressed as shown in FIG. 8C, the first and second extensions 110 and 120 suppress the side of the pimple “a” to thereby easily squeeze open the pimple “a” with a lateral force without strengthening a vertical pressure.

Also, in the case of second embodiment of the present invention, a pimple is passed through a number of grooves 112 and 121 and, a number of protrusions 111 and 122 formed in the first and second extensions 110 and 120 in a corresponding uneven shape, and pressed and suppressed therebetween alternately, so as to be easily squeezed open.

Meanwhile, the massage ball 108 in the third embodiment of the present invention is used to softly massage a place where a pimple has been squeezed open and from which a clot of sebums has been removed to thereby close a hair hole which has been opened and expanded.

### INDUSTRIAL APPLICABILITY

As described above, the pimple removal utensil provides an effect of easily squeezing open a pimple by suppressing the side of the pimple by means of the first and second extensions. In other words, the present invention can squeeze open a pimple with a minimized pressure by using a laterally suppressing force as well a simple pressing force.

Also, the pimple removal utensil according to the present invention provides an effect of removing pimples arisen in various shapes with a set of pimple removal utensils by providing a set of pimple removal utensils, which can correspondingly properly treat pimples according to the size of the pimples.

Also, the pimple removal utensil provided with a massage ball according to the present invention provides an effect of closing a hair hole by softly massaging the hair hole, which has been opened and expanded after a pimple, has been squeezed open and a sebum has been removed.

The present invention is not limited in the above-described embodiments. It is apparent to one who is skilled in the art that there are many variations and modifications without departing off the spirit of the present invention and the scope of the appended claims.

1. A pimple removal utensil (100) comprising:
a holder (101); and
a pressing portion (106) having a first extension (110) and a second extension (120) which correspond to each other and are branched off and extended from the holder (101), and a pimple insertion hole (114) formed inward the first and second extensions (110, 120).

2. The pimple removal utensil of claim 1, wherein a number of indents including a number of grooves and a number of protrusions are formed on the inner contacting surfaces of the first and second extensions, in a corresponding unevenness shape.

3. The pimple removal utensil of claim 1, wherein the bottom of the pressing portion is formed of a convex curved surface.

4. The pimple removal utensil of claim 1, wherein the interval between the first and second extensions becomes narrow gradually as it reaches from the insertion hole to the end.

5. The pimple removal utensil of claim 1, further comprising an auxiliary grip formed in the perpendicular direction from the holder.

6. The pimple removal utensil of claim 1, further comprising a ball-shaped massage ball formed on the upper portion of the holder.

7. The pimple removal utensil of claim 2, wherein the bottom of the pressing portion is formed of a convex curved surface.

8. The pimple removal utensil of claim 2, wherein the interval between the first and second extensions becomes narrow gradually as it reaches from the insertion hole to the end.
